(19) United States
(12) Patent Application Publication
(10) Pub. No.: US 2021/0355053 A1
(43) Pub. Date: Nov. 18, 2021

(54) Process for producing hydroxymethyl-alcohols

(71) Applicant: BASF SE, Ludwigshafen am Rhein (DE)

(72) Inventors: Thomas SCHAUB, Ludwigshafen (DE); Martin ERNST, Ludwigshafen (DE); Pilar CALLEJA, Heidelberg (DE); A. Stephen K. HASHMI, Heidelberg (DE)

(73) Assignee: BASF SE, Ludwigshafen am Rhein (DE)

(21) Appl. No.: 17/285,128

(22) PCT Filed: Oct. 21, 2019
(86) PCT No.: PCT/EP2019/078488

§ 371 Date: Apr. 14, 2021

(30) Foreign Application Priority Data
Oct. 31, 2018 (EP) 18203617.8

(51) Int. Cl. C07C 29/145; C07C 35/14; B01J 31/24; B01J 31/20

(52) U.S. Cl. CPC C07C 29/145; C07C 35/14; B01J 31/2409; B01J 2231/643; C07C 2601/14; B01J 2531/821; B01J 31/20

(57) ABSTRACT

A process can be used for producing an organic compound A, which contains at least one primary alcoholic hydroxy group and at least one secondary alcoholic hydroxy group. The process involves reacting a compound B, which contains at least one nitrile group and at least one ketone group, with hydrogen and water in the presence of at least one homogeneous transition metal catalyst TMC 1.

The present invention relates to a process for producing an organic compound A, which comprises at least one primary alcoholic hydroxy group and at least one secondary alcoholic hydroxy group, comprising a process step, wherein a compound B, which comprises at least one nitrile group and at least one ketone group, is reacted with hydrogen and water in the presence of at least one homogeneous transition metal catalyst TMC 1.

Hydroxymethyl-alcohols are versatile materials, especially for the use in polymer applications. For example, 5-hydroxy-1,3,3-trimethyl-cyclohexanemethanol (la) is a diol, which can be used as a monomer to prepare for example polyurethane coatings in combination with polyisocyanates as described in DE 102012003375. It can also be used as a monomer for the preparation of polyesters or polycarbonates and all other polymer applications as described in for aliphatic diols as given in Alcohols, Polyhydridic, Ulmann's encyclopedia of industrial chemistry, 2012, DOI: 10.1002/14356007.a01_305.pub2.

Currently, the only method to produce 5-hydroxy-1,3,3-trimethyl-cyclohexanemethanol is via the reduction of 5-hydroxy-1,3,3-trimethyl-cyclohexanecarbonitrile to the corresponding amine using stochiometric amounts of LiAIH₄ followed by a deamination using KOH at elevated temperatures as described in Tetrahedron Letters, 2001, 42, 8007-8010.

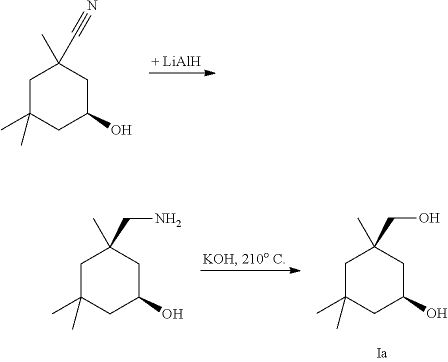

This protocol has some severe drawbacks: Stoichiometric amounts of an expensive metal-hydride has to be used for the reduction. This kind of reduction also produces stoichiometric amounts of metal waste, which must be separated and disposed. The process requires two steps, resulting in a higher complexity. The starting material is also not readily available, as it must be prepared from available Isophoronnitrile via reduction in a previous, additional step.

The reductive hydrolysis of nitriles using transition metal catalysts is described for aliphatic- as well as araliphatic nitriles by using ruthenium- or nickel catalysts whereby the nitrile is hydrogenated in the presence of water and ammonia is formed as a by-product:

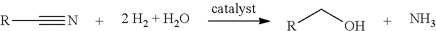

This transition metal catalyzed reductive hydrolysis of the nitrile group is described in for example in a) Catalysis Communications, 2004, 5, 237-238; b) Chinese Journal of Catalysis, 2004, 25, 611-614; c) Bulletin de la Societe chimique France, 1969,1, 126-127; d) US 5741955; e) ChemCatChem, 2017, 9, 4175-4178. But none of these documents described the synthesis of hydroxymethyl-alcohols such as the 3-hydroxymethyl-alcohol 5-hydroxy-1,3,3-trimethylcyclohexanemethanol of formula (la).

Proceeding from this prior art, it is an object of the invention to provide a technical and economically advantageous process for the production of hydroxymethyl-alcohols, such as 5-hydroxy-1,3,3-trimethyl-cyclohexanemethanol.

This object is achieved by a process for producing an organic compound A, which comprises at least one primary alcoholic hydroxy group and at least one secondary alcoholic hydroxy group, comprising a process step, wherein a compound B, which comprises at least one nitrile group and at least one ketone group, is reacted with hydrogen and water in the presence of at least one homogeneous transition metal catalyst TMC 1.

Surprisingly it was found, that when a readily available compound B, also referred to hereinafter as nitrile-ketone, is used, under the conditions of a reductive nitrile hydrolysis the ketone function is also hydrogenated and the target organic compound A, which comprises at least one primary alcoholic hydroxy group and at least one secondary alcoholic hydroxy group, is obtained in a single process step. Unlike the state of the art for the preparation of 5-hydroxy-1,3,3-trimethyl-cyclohexanemethanol, no stochiometric amount of metal hydrides are required, the byproduct is ammonia, and starting from the nitrile-ketone, the product, organic compound A, is obtained in one step compared to multiple steps in the known synthetic routes.

Preferably, the organic compound A, which comprises at least one primary alcoholic hydroxy group and at least one secondary alcoholic hydroxy group, is a compound of the formula (I)

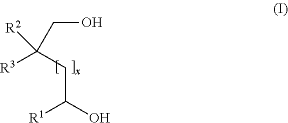

wherein
R¹ is an organic radical having from 1 ot 40 carbon atoms,
R² is hydrogen or an organic radical having from 1 ot 40 carbon atoms,
R³ is hydrogen or an organic radical having from 1 ot 40 carbon atoms,
or R¹ together with R³ or R² together with R³, together with the atoms connecting them, form a divalent organic group having from 1 to 40 carbon atoms, and
x is an integer from 1 to 10,
and the compound B, which comprises at least one nitrile group and at least one ketone group, is a compound of the formula (II)

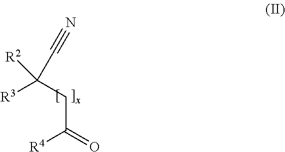

wherein
R² is hydrogen or an organic radical having from 1 ot 40 carbon atoms,
R³ is hydrogen or an organic radical having from 1 ot 40 carbon atoms,
R⁴ is an organic radical having from 1 ot 40 carbon atoms,
or R⁴ together with R³ or R² together with R³, together with the atoms connecting them, form a divalent organic group having from 1 to 40 carbon atoms. and
x is an integer from 1 to 10.

The substituents according to the present invention are, unless restricted further, defined as follows:

The term “organic radical having from 1 to 40 carbon atoms” as used in the present text refers to, for example, C₁-C₄₀-alkyl radicals, C₁-C₄₀-substituted alkyl radicals, C₁-C₁₀-fluoroalkyl radicals, C₁-C₁₂-alkoxy radicals, saturated C₃-C₂₀-heterocyclic radicals, C₆-C₄₀-aryl radicals, C₂-C₄₀-heteroaromatic radicals, C₆-C₁₀-fluoroaryl radicals, C₆-C₁₀-aryloxy radicals, silyl radicals having from 3 to 24 carbon atoms, C₂-C₂₀-alkenyl radicals, C₂-C₂₀-alkynyl radicals, C₇-C₄₀-arylalkyl radicals or C₈-C₄₀-arylalkenyl radicals. An organic radical is in each case derived from an organic compound. Thus, the organic compound methanol can in principle give rise to three different organic radicals having one carbon atom, namely methyl (H₃C—), methoxy (H₃C—O—) and hydroxymethyl (HOC(H₂)—). Therefore, the term “organic radical having from 1 to 40 carbon atoms” comprises besides alkoxy radicals for example also dialkylamino radicals, monoalkylamino radicals or alkylthio radicals.

In the present description, the term radical is used interchangeably with the term group, when defining the variables R^{x} in the presented formulas.

The term “alkyl” as used in the present text encompasses linear or singly or multiply branched saturated hydrocarbons which can also be cyclic. Preference is given to a C₁-C₁₈-alkyl radical such as methyl, ethyl, n-propyl, n-butyl, n-pentyl, n-hexyl, n-heptyl, n-octyl, n-nonyl, n-decyl, cyclopentyl, cyclohexyl, isopropyl, isobutyl, isopentyl, isohexyl, sec-butyl or tert-butyl.

The term “substituted alkyl” as used in the present text encompasses linear or singly or multiply branched saturated hydrocarbons which can also be cyclic which are monosubstituted or polysubstituted by functional groups like CN, OH, SH, NH₂, COOH, mercapto, halogen or SO₃H.

The term “alkenyl” as used in the present text encompasses linear or singly or multiply branched hydrocarbons having one or more C-C double bonds which can be cumulated or alternating.

The term “saturated heterocyclic radical” as used in the present text refers to, for example, monocyclic or polycyclic, substituted or unsubstituted aliphatic or partially unsaturated hydrocarbon radicals in which one or more carbon atoms, CH groups and/or CH₂ groups have been replaced by heteroatoms which are preferably selected from the group consisting of the elements O, S, N and P. Preferred examples of substituted or unsubstituted saturated heterocyclic radicals are pyrrolidinyl, imidazolidinyl, pyrazolidinyl, piperidyl, piperazinyl, morpholinyl, tetrahydrofuranyl, tetrahydropyranyl, tetrahydro thienyl and the like, and also methyl-, ethyl-, propyl-, isopropyl- and tert- butyl-substituted derivatives thereof.

The term “aryl” as used in the present text refers to, for example, aromatic and optionally fused polyaromatic hydrocarbon radicals which may be monosubstituted or polysubstituted by linear or branched C₁-C₁₈-alkyl, C₁-C₁₈-alkoxy, C₂-C₁₀-alkenyl, halogen, in particular fluorine, or functional groups such as COOH, hydroxy, NH₂, mercapto or SO₃H. Preferred examples of substituted and unsubstituted aryl radicals are, in particular, phenyl, pentafluorophenyl, 4-methylphenyl, 4-ethylphenyl, 4-n-propylphenyl, 4-isopropylphenyl, 4-tert-butylphenyl, 4- meth-oxyphenyl, 1-naphthyl, 9-anthryl, 9-phenanthryl, 3,5-dimethylphenyl, 3,5-di-tert-butylphenyl or 4-trifluoromethyl phenyl.

The term “heteroaromatic radical” as used in the present text refers to, for example, aromatic hydrocarbon radicals in which one or more carbon atoms or CH groups have been replaced by nitrogen, phosphorus, oxygen or sulfur atoms or combinations thereof. These may, like the aryl radicals, optionally be monosubstituted or polysubstituted by linear or branched C₁-C₁₈-alkyl, C₂-C₁₀-alkenyl, halogen, in particular fluorine, or functional groups such as COOH, hydroxy, NH₂, mercapto or SO₃H. Preferred examples are furyl, thienyl, pyrrolyl, pyridyl, pyrazolyl, imidazolyl, oxazolyl, thiazolyl, pyrimidinyl, pyrazinyl and the like, and also methyl-, ethyl-, propyl-, isopropyl- and tert-butyl-substituted derivatives thereof.

The term “arylalkyl” as used in the present text refers to, for example, aryl-comprising substituents where the corresponding aryl radical is linked via an alkyl chain to the rest of the molecule. Preferred examples are benzyl, substituted benzyl, phenethyl, substituted phenethyl and related structures.

The terms fluoroalkyl and fluoroaryl mean that at least one hydrogen atom, preferably more than one and ideally all hydrogen atoms, of the corresponding radical have been replaced by fluorine atoms. Examples of preferred fluorine-comprising radicals are trifluoromethyl, 2,2,2-trifluoroethyl, pentafluorophenyl, 4-trifluoromethylphenyl, 4-perfluoro-tert-butylphenyl and related structures.

In one embodiment of the present invention, the inventive process is characterized in that the organic compound A is a compound of the formula (I)

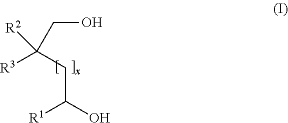

wherein
R¹ is an organic radical having from 1 ot 40 carbon atoms,
R² is hydrogen or an organic radical having from 1 ot 40 carbon atoms,
R³ is hydrogen or an organic radical having from 1 ot 40 carbon atoms,
or R¹ together with R³ or R² together with R³, together with the atoms connecting them, form a divalent organic group having from 1 to 40 carbon atoms, and
x is an integer from 1 to 10.

In one embodiment of the present invention, the inventive process is characterized in that the compound B is a compound of the formula (II)

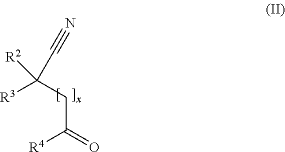

wherein
R² is hydrogen or an organic radical having from 1 ot 40 carbon atoms,
R³ is hydrogen or an organic radical having from 1 ot 40 carbon atoms,
R⁴ is an organic radical having from 1 ot 40 carbon atoms,
or R⁴ together with R³ or R² together with R³, together with the atoms connecting them, form a divalent organic group having from 1 to 40 carbon atoms. and
x is an integer from 1 to 10.

In one preferred embodiment the present invention describes a process for producing a compound of the formula (I)

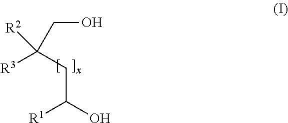

wherein
R¹ is an organic radical having from 1 ot 40 carbon atoms,
R² is hydrogen or an organic radical having from 1 ot 40 carbon atoms,
R³ is hydrogen or an organic radical having from 1 ot 40 carbon atoms,
or R¹ together with R³ or R² together with R³, together with the atoms connecting them, form a divalent organic group having from 1 to 40 carbon atoms, and
x is an integer from 1 to 10,
   comprising the process step:
a) reacting a compound of the formula (II)

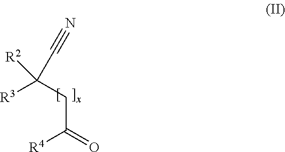

wherein R², R³ and x have the same meaning as in formula (I),
R⁴ is an organic radical having from 1 to 40 carbon atoms,
or R⁴ together with R³ or R² together with R³, together with the atoms connecting them, form a divalent organic group having from 1 to 40 carbon atoms, with hydrogen and water in the presence of at least one homogeneous transition metal catalyst TMC 1.

Compounds B, which comprise at least one nitrile group and at least one ketone group, are readily available, for example via the additions of HCN to broadly available α,β-unsaturated carbonyl compounds. The above-mentioned Isophoronnitrile is currently produced by the reaction of Isophoron with HCN as described in EP 0671384 A1. In this case x is 1 in formula I or in formula II.

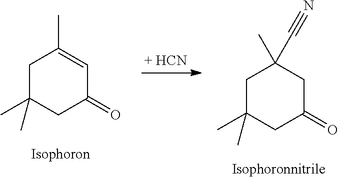

Another method to prepare nitrile-ketones according formula (I) is the addition of acrylonitrile to ketones like cyclohexanol (Organic Process Research & Development 2001, 5, 69-76) In this case x is 2 in formula I or formula II.

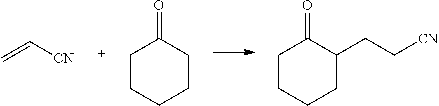

In one embodiment of the present invention, the inventive process is characterized in that the organic compound A is a compound selected from compounds of formulas Ia, Ib and Ic.

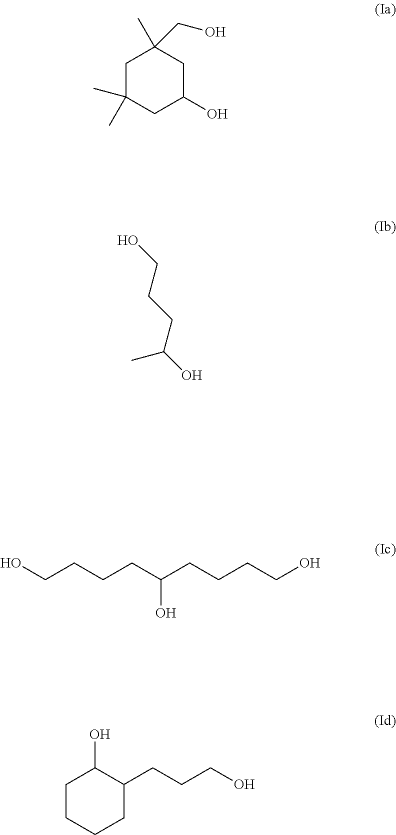

In one embodiment of the present invention, the inventive process is characterized in that the organic compound B is a compound selected from compounds of formulas IIa, IIb, IIc and IId.

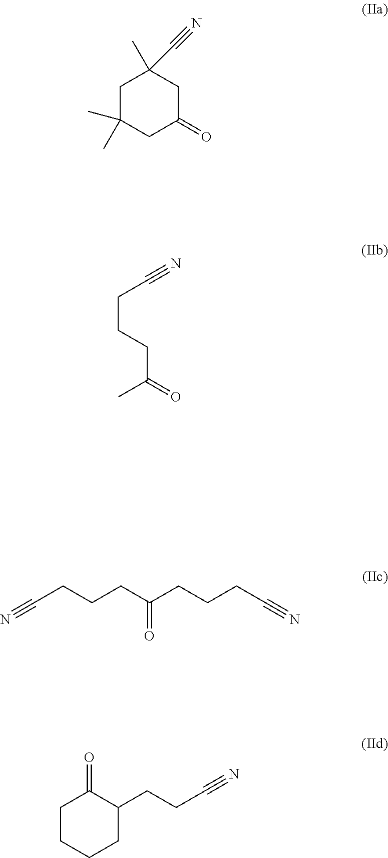

In a preferred embodiment of the invention, the nitrile-ketone is lsophoronnitrile (IIa) and the hydroxymethyl-alcohol formed is 5-hydroxy-1,3,3-trimethyl-cyclohexanemethanol (Ia).

In another preferred embodiment, the nitrile-ketone is 3-oxo-pentanenitrile (IIb) and the hydroxymethyl-alcohol formed is 1,4-pentanediol (Ib)

In another preferred embodiment, R⁴ contains also a nitrile group and the nitrile ketone is 5-oxo-nonanedinitrile (IIc) and the formed product is 1,5,8-Nonanetriol (Ic).

In another preferred embodiment, the nitrile-ketone is 2-Oxo-Cyclohexanepropanenitrile (IId) and the hydroxymethyl-alcohol formed is 2-Hydroxy-Cyclohexanepropanol (Id)

In the process of the invention, the compound B, a nitrile-ketone of formula II, is reacted with hydrogen and water in the presence of at least one homogeneous transition metal catalyst TMC 1.

The homogeneous transition metal catalyst TMC 1 comprises a transition metal selected from metals of groups 8, 9 or 10 of the periodic table of the elements according to IUPAC, such as Fe, Ru, Os, Co, Rh, Ir, Ni, Pd or Pt, preferably Ru.

In one embodiment of the present invention, the inventive process is characterized in that the homogeneous transition metal catalyst TMC 1 comprises a transition metal selected from the group consisting of metals of groups 8, 9 and 10 of the periodic table of the elements according to IUPAC, such as Fe, Ru, Os, Co, Rh, Ir, Ni, Pd or Pt, preferably ruthenium, rhodium, iridium, nickel, platinum and palladium, in particular Ru.

In one embodiment of the present invention, the inventive process is characterized in that the transition metal catalyst TMC1 is a homogeneous catalyst.

In one embodiment of the present invention, the inventive process is characterized in that the transition metal of homogeneous transition metal catalyst TMC 1 is Ru.

In one embodiment of the present invention, the inventive process is characterized in that the transition metal catalyst TMC1 is a homogeneous catalyst, wherein the transition metal of the transition metal catalyst is Ru.

The hydrogenation catalyst of the process of the invention can be employed in the form of a preformed metal complex which comprises the metal compound and one or more ligands. Alternatively, the catalytic system is formed in situ in the reaction mixture by combining a metal compound, herein also termed pre-catalyst, with one or more suitable ligands to form a catalytically active metal complex in the reaction mixture.

Suitable pre-catalysts are selected from neutral metal complexes, oxides and salts of ruthenium. Ruthenium compounds that are useful as pre-catalyst are, for example, [Ru(p-cymene)Cl₂]₂, [Ru(benzene)Cl₂]ₙ, [Ru(CO)₂Cl₂]ₙ, [Ru(CO)₃Cl₂]₂, [Ru(COD)(allyl)], [RuCl₃·H₂O], [Ru(acetylacetonate)₃], [Ru(DMSO)₄Cl₂], [Ru(PPh₃)₃Cl₂], [Ru(cyclopentadienyl)(PPh₃)₂Cl], [Ru(cyclopentadienyl)(CO)₂Cl], [Ru(cyclopentadienyl)(CO)₂H], [Ru(cyclopentadienyl)(CO)₂]₂, [Ru(pentamethylcyclopentadienyl)(CO)₂Cl], [Ru(pentamethylcyclopentadienyl)(CO)₂H], [Ru(pentamethylcyclopentadienyl)(CO)₂]₂, [Ru(indenyl)(CO)₂Cl], [Ru(indenyl)(CO)₂H], [Ru(indenyl)(CO)₂]₂, Ruthenocen, [Ru(2,2′-bipyridin)₂(Cl)₂·H₂O], [Ru(COD)(Cl)₂H]₂, [Ru(pentamethylcyclopentadienyl)(COD)Cl], [Ru₃(CO)₁₂] and [Ru(tetraphenylhydroxycyclopentadienyl)(CO)₂H].

For the hydrogenation of the process according to the present invention any complex ligands known in the art, in particular those known to be useful in ruthenium catalysed hydrogenations may be employed.

Suitable ligands of the catalytic system for the hydrogenation of the process according to the invention are, for example, mono-, bi-, tri- and tetra dentate phosphines of the formulae IV and V shown below,

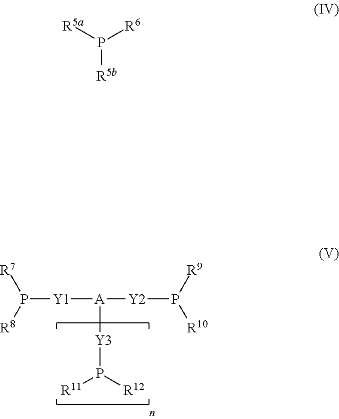

where
n is 0 or 1;
R^{5a} to R¹² are, independently of one another, unsubstituted or at least monosubstituted C₁-C₁₀-alkyl, C₁-C₄-alkyldiphenylphosphine (—C₁-C₄-alkyl-P(phenyl)₂), C₃-C₁₀-cycloalkyl, C₃-C₁₀-heterocyclylcomprising at least one heteroatom selected from N, O and S, C₅-C₁₄-aryl or C₅-C₁₀-heteroaryl comprising at least one heteroatom selected from N, O and S,
where the substituents are selected from the group consisting of: F, CI, Br, OH, CN, NH₂ and C₁-C₁₀-alkyl;

A is
i) a bridging group selected from the group unsubstituted or at least monosubstituted N, O, P, C₁-C₆-alkane, C₃-C₁₀-cycloalkane, C₃-C₁₀-heterocycloalkane comprising at least one heteroatom selected from N, O and S, C₅-C₁₄-aromatic and C₅-C₆-heteroaromatic comprising at least one heteroatom selected from N, O and S, where the substituents are selected from the group consisting of:
C₁-C₄-alkyl, phenyl, F, Cl, Br, OH, OR¹⁶, NH₂, NHR¹⁶ or N(R¹⁶)₂,
where R¹⁶ is selected from C₁-C₁₀-alkyl and C₅-C₁₀-aryl; pr
ii) a bridging group of the formula (VI) or (VII):

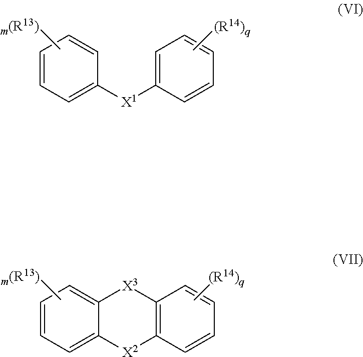

m, q are, independently of one another, 0, 1, 2, 3 or 4;
R¹³, R¹⁴ are, independently of one another, selected from the group C₁-C₁₀-alkyl,
F, Cl, Br, OH, OR¹⁵, NH₂, NHR¹⁵ and N(R¹⁵)₂,
   where R¹⁵ is selected from C₁-C₁₀-alkyl and C₅-C₁₀-aryl;
X¹, X² are, independently of one another, NH, O or S;
X³ is a bond, NH, NR¹⁶, O, S or CR¹⁷R¹⁸;
R16 is unsubstituted or at least monosubstituted C₁-C₁₀-alkyl, C₃-C₁₀-cycloalkyl, C₃-C₁₀-heterocyclylcomprising at least one heteroatom selected from N, O and S, C₅-C₁₄-aryl or C₅-C₁₀-heteroaryl comprising at least one heteroatom selected from N, O and S,
where the substituents are selected from the group consisting of: F, Cl, Br, OH, CN, NH₂ and C₁-C₁₀-alkyl;
R¹⁷, R¹⁸ are, independently of one another, unsubstituted or at least monosubstituted C₁-C₁₀-alkyl, C₁-C₁₀-alkoxy, C₃-C₁₀-cycloalkyl, C₃-C₁₀-cycloalkoxy, C₃-C₁₀-heterocyclylcomprising at least one heteroatom selected from N, O and S, C₅-C₁₄-aryl, C₅-C₁₄-aryloxy or C₅-C₁₀-heteroaryl comprising at least one heteroatom selected from N, O and S,
where the substituents are selected from the group consisting of: F, Cl, Br, OH, CN, NH₂ and C₁-C₁₀-alkyl;
Y¹, Y², Y³ are, independently of one another, a bond, unsubstituted or at least monosubstituted methylene, ethylene, trimethylene, tetramethylene, pentamethylene or hexamethylene,
where the substituents are selected from the group consisting of: F, Cl, Br, OH, OR¹⁵, CN, NH₂, NHR¹⁵, N(R¹⁵)₂ and C₁-C₁₀-alkyl,
where R¹⁵is selected from C₁-C₁₀-alkyl and C₅-C₁₀-aryl.

A is a bridging group. For the case that A is selected from the group unsubstituted or at least monosubstituted C₁-C₆-alkane, C₃-C₁₀-cycloalkane, C₃-C₁₀-heterocycloalkane, C₅-C₁₄-aromatic and C₅-C₆-heteroaromatic for the case (n=0), two hydrogen atoms of the bridging group are replaced by bonds to the adjacent substituents Y¹ and Y². For the case (n=1), three hydrogen atoms of the bridging group are replaced by three bonds to the adjacent substituents Y¹, Y² and Y³.

For the case that A is P (phosphorus), the phosphorus forms for the case (n=0) two bonds to the adjacent substituents Y¹ and Y² and one bond to a substituent selected from the group consisting of C₁-C₄-alkyl and phenyl. For the case (n=1), the phosphorus forms three bonds to the adjacent substituents Y¹, Y² and Y³.

For the case that A is N (nitrogen), the nitrogen for the case (n=0) forms two bonds to the adjacent substituents Y¹ and Y² and one bond to a substituent selected from the group consisting of C₁-C₄-alkyl and phenyl. For the case (n=1), the nitrogen forms three bonds to the adjacent substituents Y¹, Y² and Y³.

For the case that A is O (oxygen), n=0. The oxygen forms two bonds to the adjacent substituents Y¹ and Y².

Preference is given to complex catalysts which comprise at least one element selected from ruthenium and iridium.

In a preferred embodiment, the process according to the invention is carried out in the presence of at least one complex catalyst which comprises at least one element selected from the groups 8, 9 and 10 of the Periodic Table of the Elements and also at least one phosphorus donor ligand of the general formula (V), where
n is 0 or 1;
R⁷ to R¹² are, independently of one another, unsubstituted C₁-C₁₀-alkyl, C₃-C₁₀-cycloalkyl, C₃-C₁₀-heterocyclylcomprising at least one heteroatom selected from N, O and S, C₅-C₁₄-aryl or C₅-C₁₀-heteroaryl comprising at least one heteroatom selected from N, O and S;
A is
   i) a bridging group selected from the group unsubstituted C₁-C₆-alkane, C₃-C₁₀-cycloalkane, C₃-C₁₀-heterocycloalkane comprising at least one heteroatom selected from N, O and S, C₅-C₁₄-aromatic and C₅-C₆-heteroaromatic comprising at least one heteroatom selected from N, O and S; pr
   ii) a bridging group of the formula (VI) or (VII):

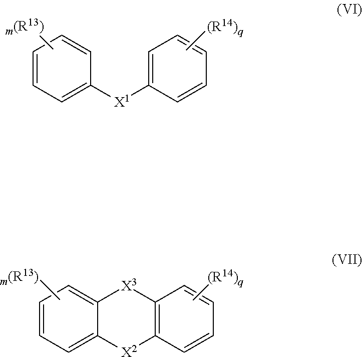

m, q are, independently of one another, 0, 1, 2, 3 or 4;
R¹³, R¹⁴ are, independently of one another, selected from the group C₁-C₁₀-alkyl, F, Cl, Br, OH, OR¹⁵, NH₂, NHR¹⁵ and N(R¹⁵)₂,
   where R¹⁵ is selected from C₁-C₁₀-alkyl and C₅-C₁₀-aryl;
X¹, X² are, independently of one another, NH, O or S;
X³ is a bond, NH, NR¹⁶, O, S or CR¹⁷R¹⁸;
R¹⁶ is unsubstituted C₁-C₁₀-alkyl, C₃-C₁₀-cycloalkyl, C₃-C₁₀-heterocyclyl comprising at least one heteroatom selected from N, O and S, C₅-C₁₄-aryl or C₅-C₁₀-heteroaryl comprising at least one heteroatom selected from N, O and S;
R^{17,} R¹⁸ are, independently of one another, unsubstituted C₁-C₁₀-alkyl, C₁-C₁₀-alkoxy, C₃-C₁₀-cycloalkyl, C₃-C₁₀-cycloalkoxy, C₃-C₁₀-heterocyclyl comprising at least one heteroatom selected from N, O and S, C₅-C₁₄-aryl, C₅-C₁₄-aryloxy or C₅-C₁₀-heteroaryl comprising at least one heteroatom selected from N, O and S;
Y¹, Y², Y³ are, independently of one another, a bond, unsubstituted methylene, ethylene, trimethylene, tetramethylene, pentamethylene or hexamethylene.

In a further preferred embodiment, the process according to the invention is carried out in the presence of at least one complex catalyst which comprises at least one element selected from groups 8, 9 and 10 of the Periodic Table of the Elements and also at least one phosphorus donor ligand of the general formula (VIII),

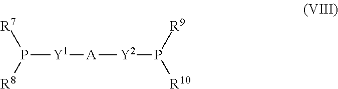

where
R⁷ to R¹⁰ are, independently of one another, unsubstituted or at least monosubstituted C₁-C₁₀-alkyl, C₁-C₄-alkyldiphenylphosphine (—C₁-C₄-alkyl-P(phenyl)₂), C₃-C₁₀-cycloalkyl, C₃-C₁₀-heterocyclyl comprising at least one heteroatom selected from N, O and S, C₅-C₁₄-aryl or C₅-C₁₀-heteroaryl comprising at least one heteroatom selected from N, O and S,
where the substituents are selected from the group consisting of: F, Cl, Br, OH, CN, NH₂ and C₁-C₁₀-alkyl;
A is
   i) a bridging group selected from the group unsubstituted or at least monosubstituted N, O, P, C₁-C₆-alkane, C₃-C₁₀-cycloalkane, C₃-C₁₀-heterocycloalkane comprising at least one heteroatom selected from N, O and S, C₅-C₁₄-aromatic and C₅-C₆-heteroaromatic comprising at least one heteroatom selected from N, O and S,
where the substituents are selected from the group consisting of: C₁-C₄-alkyl, phenyl, F, Cl, Br, OH, OR¹⁵, NH₂, NHR¹⁵ or N(R¹⁵)₂,
where R¹⁵ is selected from C₁-C₁₀-alkyl and C₅-C₁₀-aryl; or
   ii) a bridging group of the formula (VI) or (VII):

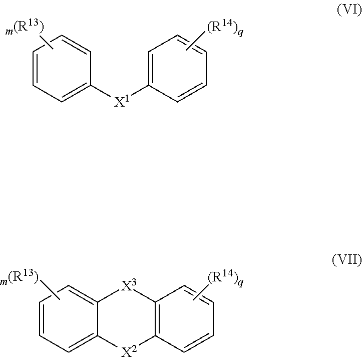

m, q are, independently of one another, 0, 1, 2, 3 or 4;
R¹³, R¹⁴ are, independently of one another, selected from the group C₁-C₁₀-alkyl, F, Cl, Br, OH, OR¹⁵, NH₂, NHR¹⁵ and N(R¹⁵)₂,
   where R¹⁵ is selected from C₁-C₁₀-alkyl and C₅-C₁₀-aryl;
X¹, X² are, independently of one another, NH, O or S,
X³ is a bond, NH, NR¹⁶, O, S or CR¹⁷R¹⁸;
R¹⁶ is unsubstituted or at least monosubstituted C₁-C₁₀-alkyl, C₃-C₁₀-cycloalkyl, C₃-C₁₀-heterocyclyl comprising at least one heteroatom selected from N, O and S, C₅-C₁₄-aryl or C₅-C₁₀-heteroaryl comprising at least one heteroatom selected from N, O and S,
   where the substituents are selected from the group consisting of: F, Cl, Br, OH, CN, NH₂ and C₁-C₁₀-alkyl;
R¹⁷, R¹⁸ are, independently of one another, unsubstituted or at least monosubstituted C₁-C₁₀-alkyl, C₁-C₁₀-alkoxy, C₃-C₁₀-cycloalkyl, C₃-C₁₀-cycloalkoxy, C₃-C₁₀-heterocyclylcomprising at least one heteroatom selected from N, O and S, C₅-C₁₄-aryl, C₅-C₁₄-aryloxy or C₅-C₁₀-heteroaryl comprising at least one heteroatom selected from N, O and S,
   where the substituents are selected from the group consisting of: F, Cl, Br, OH, CN, NH₂ and C₁-C₁₀-alkyl;
Y₁, Y₂ are, independently of one another, a bond, unsubstituted or at least monosubstituted methylene, ethylene, trimethylene, tetramethylene, pentamethylene or hexamethylene,
   where the substituents are selected from the group consisting of: F, Cl, Br, OH, OR¹⁵, CN, NH_{2,} NHR¹⁵, N(R¹⁵)₂ and C₁-C₁₀-alkyl,
   where R¹⁵is selected from C₁-C₁₀-alkyl and C₅-C₁₀-aryl.

In a further preferred embodiment, the process according to the invention is carried out in the presence of at least one complex catalyst which comprises at least one element selected from groups 8, 9 and 10 of the Periodic Table of the Elements and also at least one phosphorus donor ligand of the general formula (IX),

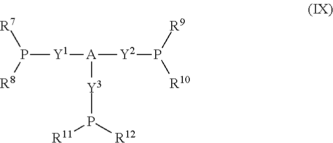

where
R⁷ to R¹² are, independently of one another, unsubstituted or at least monosubstituted C₁-C₁₀-alkyl, C₁-C₄-alkyldiphenylphosphine, C₃-C₁₀-cycloalkyl, C₃-C₁₀-heterocyclyl comprising at least one heteroatom selected from N, O and S, C₅-C₁₄-aryl or C₅-C₁₀-heteroaryl comprising at least one heteroatom selected from N, O and S,
   where the substituents are selected from the group consisting of: F, Cl, Br, OH, CN, NH₂ and C₁-C₁₀-alkyl;
A is a bridging group selected from the group unsubstituted or at least monosubstituted N, P, C₁-C₆-alkane, C₃-C₁₀-cycloalkane, C₃-C₁₀-heterocycloalkane comprising at least one heteroatom selected from N, O and S, C₅-C₁₄-aromatic and C₅-C₆-heteroaromatic comprising at least one heteroatom selected from N, O and S,
   where the substituents are selected from the group consisting of: C₁-C₄-alkyl, phenyl, F, Cl, Br, OH, OR¹⁵, NH₂, NHR¹⁵ or N(R¹⁵)₂,
   where R¹⁵ is selected from C₁-C₁₀-alkyl and C₅-C₁₀-aryl;
Y¹, Y², Y³ are, independently of one another, a bond, unsubstituted or at least monosubstituted methylene, ethylene, trimethylene, tetramethylene, pentamethylene or hexamethylene,
   where the substituents are selected from the group consisting of: F, Cl, Br, OH, OR¹⁵, CN, NH₂, NHR¹⁵, N(R¹⁵)₂ and C₁-C₁₀-alkyl,
   where R¹⁵ is selected from C₁-C₁₀-alkyl and C₅-C₁₀-aryl.

In a further preferred embodiment, the process according to the invention is carried out in the presence of at least one complex catalyst which comprises at least one element selected from groups 8, 9 and 10 of the Periodic Table of the Elements and also at least one phosphorus donor ligand of the general formula (VIII), where
R⁷ to R¹⁰ are, independently of one another, methyl, ethyl, isopropyl, tert-butyl, cyclopentyl, cyclohexyl, phenyl, or mesityl;
A is
   i)a bridging group selected from the group methane, ethane, propane, butane, cyclohexane, benzene, napthalene and anthracene; or
   ii) a bridging group of the formula (X) or (XI):

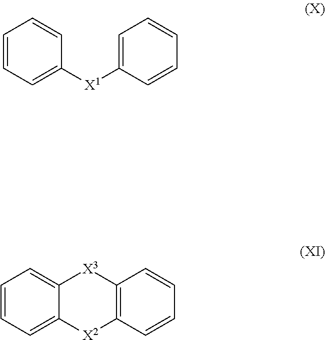

X¹, X² are, independently of one another, NH, O or S;
X³ is a bond, NH, O, S or CR¹⁷1R¹⁸;
R¹⁷, R¹⁸ are, independently of one another, unsubstituted C₁-C₁₀-alkyl;
Y¹, Y² are, independently of one another, a bond, methylene or ethylene.

In a particularly preferred embodiment, the process according to the invention is carried out in the presence of at least one complex catalyst which comprises at least one element selected from groups 8, 9 and 10 of the Periodic Table of the Elements and also at least one phosphorus donor ligand of the general formula (XII) or (XIII),

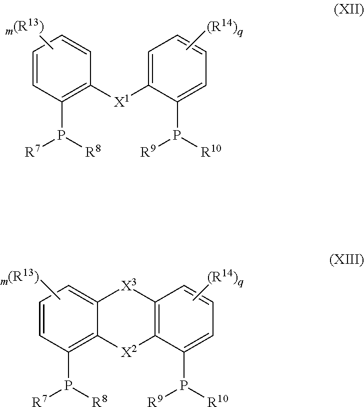

where for m, q, R⁷, R⁸, R⁹, R¹⁰, R¹³, R¹⁴, X¹, X² and X³, the definitions and preferences listed above are applicable.

In an embodiment, the process according to the invention is carried out in the presence of at least transition metal one complex catalyst and monodentate ligands of the formula IV are preferred herein are those in which R^{5a}, R^{5b} and R⁶ are each phenyl or alkyl optionally carrying 1 or 2 C₁-C₄-alkyl substituents and those in which R⁷, R⁸ and R⁹ are each C₅-C₈-cycloalkyl or C₂-C₁₀-alkyl, in particular linear unbranched n-C₂-C₁₀-alkyl. The groups R^{5a} to R⁶ may be different or identical. Preferably the groups R^{5a} to R⁶ are identical and are selected from the substituents mentioned herein, in particular from those indicated as preferred. Examples of preferable monodentate ligands IV are triphenylphosphine (TPP), Triethylphosphine, tri-n-butylphosphine, tri-n-octylphosphine and tricyclohexylphosphine.

In another embodiment, the process according to the invention is carried out in the presence of at least transition metal one complex catalyst and at least one phosphorus donor ligand selected from the group 1,2-bis(diphenylphosphino)ethane (dppe), 1,2-bis(diphenylphosphino)propane (dppp), 1,2-bis(diphenylphosphino)butane (dppb), 2,3-bis(dicyclohexylphosphino)ethane (dcpe), 4,5-bis(diphenylphosphino)-9,9-dimethylxanthene (xantphos), bis(2-diphenylphosphinoethyl)phenylphosphine and 1,1,1-tris(diphenylphosphinomethyl)ethane (triphos).

In a further particularly preferred embodiment, the process according to the invention is carried out in the presence of a complex catalyst which comprises ruthenium and at least one phosphorus donor ligand selected from the group 4,5-bis(diphenylphosphino)-9,9-dimethylxanthene (xantphos), bis(2-diphenylphosphinoethyl)phenylphosphine and 1,1,1-tris(diphenylphosphinomethyl)ethane (triphos).

In a further particularly preferred embodiment, the process according to the invention is carried out in the presence of a complex catalyst which comprises iridium and also at least one phosphorus donor ligand selected from the group 4,5-bis(diphenylphosphino)-9,9-dimethylxanthene (xantphos), bis(2-diphenylphosphinoethyl)phenylphosphine and 1,1,1-tris(diphenylphosphinomethyl)ethane (triphos).

Within the context of the present invention, C₁-C₁₀-alkyl is understood as meaning branched, unbranched, saturated and unsaturated groups. Preference is given to alkyl groups having 1 to 6 carbon atoms (C₁-C₆-alkyl). More preference is given to alkyl groups having 1 to 4 carbon atoms (C₁-C₄-alkyl).

Examples of saturated alkyl groups are methyl, ethyl, n-propyl, isopropyl, n-butyl, isobutyl, secbutyl, tert-butyl, amyl and hexyl.

Examples of unsaturated alkyl groups (alkenyl, alkynyl) are vinyl, allyl, butenyl, ethynyl and propynyl.

The C₁-C₁₀-alkyl group can be unsubstituted or substituted with one or more substituents selected from the group F, Cl, Br, hydroxy (OH), C₁-C₁₀-alkoxy, C₅-C₁₀-aryloxy, C₅-C₁₀-alkylaryloxy, C₅-C₁₀-heteroaryloxy comprising at least one heteroatom selected from N, O, S, oxo, C₃-C₁₀-cycloalkyl, phenyl, C₅-C₁₀-heteroaryl comprising at least one heteroatom selected from N, O, S, C₅-C₁₀-heterocyclyl comprising at least one heteroatom selected from N, O, S, naphthyl, amino, C₁-C₁₀-alkylamino, C₅-C₁₀-arylamino, C₅-C₁₀-heteroarylamino comprising at least one heteroatom selected from N, O, S, C₁-C₁₀-dialkylamino, C₁₀-C₁₂-diarylamino, C₁₀-C₂₀-alkylarylamino, C₁-C₁₀-acyl, C₁-C₁₀-acyloxy, NO₂, C₁-C₁₀-carboxy, carbamoyl, carboxamide, cyano, sulfonyl, sulfonylamino, sulfinyl, sulfinylamino, thiol, C₁-C₁₀-alkylthiol, C₅-C₁₀-arylthiol or C₁-C₁₀-alkylsulfonyl.

The above definition for C₁-C₁₀-alkyl applies correspondingly to C₁-C₃₀-alkyl and to C₁-C₆-alkane.

C₃-C₁₀-cycloalkyl is understood in the present case as meaning saturated, unsaturated monocyclic and polycyclic groups. Examples of C₃-C₁₀-cycloalkyl are cyclopropyl, cyclobutyl, cyclopentyl, cyclohexyl or cycloheptyl. The cycloalkyl groups can be unsubstituted or substituted with one or more substituents as has been defined above in connection with the group C₁-C₁₀-alkyl.

The active hydrogenation catalyst can be generated in situ in the reaction mixture by adding the ligands to the above-mentioned precursors. The molar ratio between the transition metal and the ligand is in the range of 2:1 to 1:50, preferable in the range of 1:1 to 1:10 most preferer ably in the range of 1:2 to 1:5.

In addition to the one or more ligands selected from the groups of ligands described above the catalytic system of the inventive process may also include at least one further ligand which is selected from halides, amides, carboxylates, acetylacetonate, aryl- or alkylsufonates, hydride, CO, olefins, dienes, cycloolefines, nitriles, aromatics and heteroaromatics, ethers, PF₃, phospholes, phosphabenzenes, and mono-, di- and polydentate phosphinite, phosphonite, phosphoramidite and phosphite ligands. Preferably the catalyst also contains CO as a ligand.

The active catalyst can also be preformed in a dedicated synthetic step. Appropriate preformed catalysts can be [Ru(PPh₃)₃(CO)(H)Cl], [Ru(PPh₃)₃(CO)Cl₂], [Ru(PPh₃)₃(CO)(H)2], [Ru(binap)(Cl)₂], [Ru(PMe₃)₄(H)₂], [Ru(PEt₃)₄(H)₂], [Ru(Pn-Pr₃)₄(H)2], [Ru(Pn-Bu₃)₄(H)₂], [Ru(Pn-Octyl₃)₄(H)₂], [Ru(Pn-Bu₃)₄(H)₂], [Ru(PnOctyl₃)₄(H)₂], [Ru(PPh₃)₃(CO)(H)Cl] and [Ru(PPh₃)3(CO)(H)₂], preferably [Ru(PPh₃)₃(CO)(H)Cl], [Ru(PPh₃)₃(CO)C₁₂], [Ru(PPh₃)₃(CO)(H)₂ and most preferably the active catalyst is [Ru(PPh₃)₃(CO)(H)Cl].

In one embodiment of the present invention, the inventive process is characterized in that the homogeneous transition metal catalyst TMC 1 is selected from the group consisting of [Ru(PPh₃)₃(CO)(H)Cl], [Ru(PPh₃)₃(CO)Cl₂], [Ru(PPh₃)₃(CO)(H)₂], [Ru(binap)(Cl)₂], [Ru(PMe₃)₄(H)₂], [Ru(PEt₃)₄(H)₂], [Ru(Pn-Pr₃)₄(H)₂], [Ru(Pn-Bu₃)₄(H)₂], [Ru(Pn-Octyl₃)₄(H)₂], [Ru(Pn-Bu₃)₄(H)₂], [Ru(PnOctyl₃)₄(H)₂], [Ru(PPh₃)₃(CO)(H)Cl] and [Ru(PPh₃)₃(CO)(H)₂], preferably [Ru(PPh₃)₃(CO)(H)Cl], [Ru(PPh₃)₃(CO)Cl₂], [Ru(PPh₃)₃(CO)(H)₂ and most preferably [Ru(PPh₃)₃(CO)(H)Cl].

If a preformed active catalyst is used, it can also be beneficial to add additional ligand of the formula IV or V to the reaction mixture.

In the inventive process the amount of transition metal catalyst TMC1 used based on the amount of compound B, preferably the nitrile-ketones according to formula II, can be varied in a wide range. Usually the homogeneous transition metal catalyst TMC 1 is used in a substoichiometric amount relative to compound B. Typically, the amount of homogeneous transition metal catalyst TMC 1 is not more than 50 mol %, frequently not more than 20 mol % and in particular not more than 10 mol % or not more than 5 mol %, based on the amount of compound B. An amount of homogeneous transition metal catalyst TMC 1 of from 0.001 to 50 mol %, frequently from 0.001 mol % to 20 mol % and in particular from 0.005 to 5 mol %, based on the amount of compound B is preferably used in the process of the invention. Preference is given to using an amount of transition metal catalyst of from 0.01 to 5 mol %. All amounts of transition metal complex catalyst indicated are calculated as transition metal and based on the amount of compound B.

In one embodiment of the present invention, the inventive process is characterized in that the transition metal complex catalyst TMC1 is used in an amount of 0.001 mol % to 20 mol %, calculated as transition metal and based on the amount of compound B used in the process.

The reaction of compound B with hydrogen and water can principally be performed according to all processes known to a person skilled in the art which are suitable for the reaction of nitrileketones according to formula II with H₂ in the presence of water.

The hydrogen (H₂) used for the reduction reaction can be used in pure form or, if desired, also in the form of mixtures with other, preferably inert gases, such as nitrogen or argon. Preference is given to using H₂ in undiluted form.

The reaction is typically carried at a H₂ pressure in the range from 0.1 to 400 bar, preferably in the range from 10 to 200 bar, more preferably in the range from 20 to 180 bar.

In one embodiment of the present invention, the inventive process is characterized in that the reaction between compound B, water and hydrogen is performed at a pressure in the range from 20 to 180 bar.

The reaction can principally be performed continuously, semi-continuously or discontinuously. Preference is given to a continuous process.

The reaction can principally be performed in all reactors known to a person skilled in the art for this type of reaction and who will therefore select the reactors accordingly. Suitable reactors are described and reviewed in the relevant prior art, e.g. appropriate monographs and reference works such as mentioned in U.S. Pat. No. 6,639,114 B2, column 16, line 45-49. Preferably, for the reaction an autoclave is employed which may have an internal stirrer and an internal lining.

The composition obtained in the reductive nitrile hydrolysis of the present invention comprises an organic compound A, preferably the hydroxymethyl-alcohols according to formula I as described above.

The inventive process can be performed in a wide temperature range. Preferably the reaction is performed at a temperature in the range from 20° C. to 200° C., more preferably in the range from 50° C. to 180° C., in particular in the range from 100° C. to 170° C.

In one embodiment of the present invention, the inventive process is characterized in that the reaction between compound B, water and hydrogen is performed at a temperature in the range from 50° C. to 180° C.

The reductive nitrile hydrolysis and ketone hydrogenation is carried out in the presence of water. The reaction can be run in water as solvent but also in combination with a solvent. Use of water-solvent mixtures is preferred in the reductive nitrile hydrolysis. Suitable solvents are selected from aliphatic hydrocarbons, aromatic hydrocarbons, ethers or alcohols and mixtures thereof. Preferred solvents are
aliphatic hydrocarbons such as pentane, hexane, heptane, octane or cyclohexane;
aromatic hydrocarbons such as benzene, toluene, xylenes, ethylbenzene, mesitylene or benzotrifluoride;
ethers such as dioxane, tetrahydrofuran, 2-methyl-tetrahydrofuran, diethyl ether, dibutyl ether, methyl t-butyl ether, diisopropyl ether, dimethoxyethane, or diethylene glycol dimethyl ether and other glymes (ethers of various oligomers of propyleneglycols and ethyleneglycols);
alcohols such as methanol, ethanol, 2-propanol, 1-butanol, iso-butanol, tert-butanol, methoxyethanol

Preference is given to using a solvent selected from the group of solvents consisting of dioxane, tetrahydrofuran, glymes, methanol and ethanol.

In one embodiment of the present invention, the inventive process is characterized in that the reaction between compound B, water and hydrogen is performed in the presence of a solvent selected from the group of solvents consisting of dioxane, tetrahydrofuran, glymes, methanol and ethanol.

If desired, mixtures of two or more of the afore-mentioned solvents can also be used.

The molar ratio of water to solvent, when additional solvents are used, is in the range between 50:1 to 1:50, preferably between 2:1 to 1:30, most preferably 2:1 to 1:10.

Alternatively, the process of the invention can be carried out in the absence of any of the above-mentioned organic solvent, so-called neat conditions, preferably in the presence of the organic compound A, preferably the hydroxymethyl-alcohols according to formula I as described above, as solvent together with water.

The composition obtained in the inventive process, the reductive nitrile hydrolysis and ketone hydrogenation, comprises the organic compound A, preferably 3- or 4-hydroxymethyl-alcohols according to formula I. The work-up of the reaction mixture of the inventive process and the isolation of the organic compound A are carried out in a customary manner, for example by filtration, an extractive work-up or by a distillation, for example under reduced pressure. The organic compound A may be obtained in sufficient purity by applying such measures or a combination thereof, obviating additional purification steps. Alternatively, further purification can be accomplished by methods commonly used in the art, such as chromatography.

In one embodiment of the present invention, the inventive process is characterized in that the organic compound A, preferably the hydroxymethyl-alcohol according to formula I is separated from the transition metal catalyst after the reductive nitrile hydrolysis via distillation.

The distillation residue usually still comprises the transition metal catalyst in an active form, that can be reused in a new reductive nitrile hydrolysis and ketone hydrogenation step, that is a new process step a. As long as the distillation conditions, in particular the temperature treatment, are not too harsh, the transition metal catalyst remains active.

In one embodiment of the present invention, the inventive process is characterized in that the homogeneous transition metal catalyst TMC 1 is recycled by removing compound A and other volatile compounds of the reaction mixture via distillation.

The present invention offers an economical process for producing hydroxymethyl-alcohols from readily available nitrile-ketones in a single process step.

The invention is illustrated by the examples which follow, but these do not restrict the invention.

Figures in percent are each based on % by weight, unless explicitly stated otherwise.

### GENERAL

All chemicals and solvents were purchased from Sigma-Aldrich or ABCR and used without further purification. Analytical thin layer chromatography (TLC) was performed on pre-coated Macherey-Nagel POLYGRAM° SIL G/UV₂₅₄ polyester sheets. Visualization was achieved using potassium permanganate stain [KMnO₄ (10 g), K₂CO₃ (65 g), and aqueous NaOH solution (1 N, 15 mL) in water (1000 mL)] followed by heating. Column chromatography was carried out on Aldrich silica gel (60 Å, 70-230 mesh, 63-200 μm). ¹H and ¹³C NMR spectra were recorded on either a Bruker Avance III 300, Bruker Avance III 400, or a Bruker Avance III 500 spectrometer at ambient temperature. Chemical shifts δ are reported in ppm relative to either the residual solvent or tetramethylsilane (TMS). The multiplicities are reported as: s=singlet, bs=broad singlet, d=doublet, t=triplet, q=quartet, m=multiplet, td=triplet of doublets, tt=triplet of triplets.

### Example 1

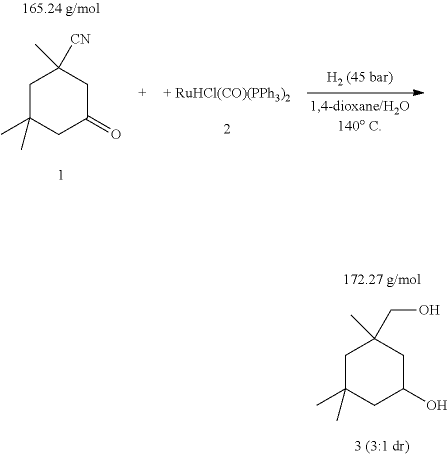

| Reagents | MW [g/mol] | equiv | mmol | grams (mg) |
|---|---|---|---|---|
| 1 | 165.24 | 1 | 1 | 165.2 |
| 2 | 952.41 | 0.05 | 0.05 | 47.6 |

Procedure: A ca. 80 mL Parr autoclave was charged with RuHCl(CO)(PPh₃)₃ (47.6 mg, 0.05 mmol), the nitrile (165.2 mg, 1 mmol), 1,4-dioxane (12.0 mL) and water (12.0 mL) under air. The mixture was degassed gently with argon. After closing the reaction vessel, the system was purged first with nitrogen (3×) and then with hydrogen (3×). Finally, the autoclave was pressurized with hydrogen (45 bar) and heated at 140° C. Stirred under these conditions for 22 h. Note: At this temperature the internal pressure rises up to 55 bar. Then, the reaction was allowed to cool down to rt and was depressurized carefully. To the crude was added brine (10 mL) and the organic phase was extracted with EtOAc (3×30 mL), washed with brine and dried over Na₂SO₄. Filtered through a short cotton pad and concentrated under vacuum. The crude was purified by flash column chromatography over SiO₂ using Hexane/EtOAc/Acetone (1:1:0.1) as eluent. The product was isolated as a 3:1 mixture of diastereomers. Yellow oil (136.8 mg, 80% yield). Major isomer: ¹H NMR (300 MHz, CDCl₃) δ3.98 (tt, J=11.4, 4.1 Hz, 1 H), 3.23 (s, 2 H), 1.81-1.72 (m, 2 H), 1.71-1.62 (m, 2 H), 1.15 (s, 2 H), 1.04 (s, 3 H), 1.03 (s, 3 H), 0.96 (s, 3 H). ¹³C NMR (75 MHz, CDCl₃) δ75.1, 65.9, 49.0, 45.9, 43.2, 37.6, 35.2, 32.5, 28.4, 23.2. Minor isomer: ¹H NMR (300 MHz, CDCl₃) δ3.87 (tt, J=11.4, 4.1 Hz, 1 H), 3.51 (s, 2 H), 1.96-1.84 (m, 2 H), 1.52-1.44 (m, 2 H), 1.11 (s, 2 H), 1.07 (s, 3 H), 0.99 (s, 3H+3H). Note: Some of the ¹H NMR signals are partially overlapped with the signals of the major isomer. ¹³C NMR (75 MHz, CDCl₃) δ69.1, 65.7, 48.7, 46.2, 44.0, 37.8, 35.2, 32.3, 29.3, 28.0.

### Example 2

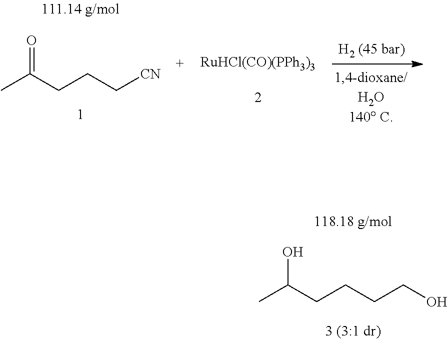

| Reagents | MW [g/mol] | equiv | mmol | grams (mg) |
|---|---|---|---|---|
| 1 | 111.14 | 1 | 1 | 111.1 |
| 2 | 952.41 | 0.05 | 0.05 | 47.6 |

Procedure: A ca. 40 mL Premex autoclave was charged with RuHCl(CO)(PPh₃)₃, the nitrile, 1,4-dioxane (6.0 mL) and water (6.0 mL) under air. The mixture was degassed gently with argon. After closing the reaction vessel, the system was purged first with nitrogen (3×) and then with hydrogen (3×). Finally, the autoclave was pressurized with hydrogen (45 bar) and heated at 140° C. Stirred under these conditions for 22 h. Note: At this temperature the internal pressure rises up to 55 bar. Then, the reaction was allowed to cool down to rt and was depressurized carefully. To the crude was added brine (10 mL) and the organic phase was extracted with EtOAc (3×30 mL), washed with brine and dried over Na₂SO₄. Filtered through a short cotton pad and concentrated under vacuum. The crude was purified by flash column chromatography over SiO₂ using Hexane/EtOAc (gradient from 40% to 70%) as eluent. The product was isolated as a brown oil (47.7 mg, 40% yield).

¹H NMR (400 MHz, CDCl₃) δ3.85-3.77 (m, 1 H), 3.66 (t, J=6.4 Hz, 2 H), 1.66 (bs, 2 H), 1.63-1.38 (m, 6 H), 1.19 (d, J=6.2 Hz, 3 H). ¹³C NMR (101 MHz, CDCl₃) δ68.2, 62.9, 39.0, 32.7, 23.7, 22.1.

### Example 3

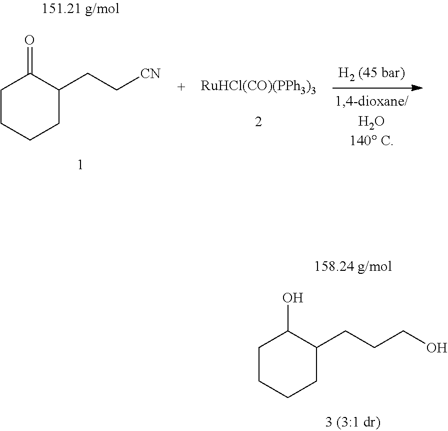 [SMILES: N#CCCC1CCCCC1=O ; "RuHCl(CO)(PPh3)3" ; OCCCC1CCCCC1O]

| Reagents | MW [g/mol] | equiv | mmol | grams (mg) |
|---|---|---|---|---|
| 1 | 151.21 | 1 | 1 | 151.2 |
| 2 | 952.41 | 0.05 | 0.05 | 47.6 |

Procedure: A ca. 40 mL Premex autoclave was charged with RuHCl(CO)(PPh₃)₃, the nitrile, 1,4-dioxane (6.0 mL) and water (6.0 mL) under air. The mixture was degassed gently with argon. After closing the reaction vessel, the system was purged first with nitrogen (3×) and then with hydrogen (3×). Finally, the autoclave was pressurized with hydrogen (45 bar) and heated at 140° C. Stirred under these conditions for 22 h. Note: At this temperature the internal pressure rises up to 55 bar. Then, the reaction was allowed to cool down to rt and was depressurized carefully. To the crude was added brine (10 mL) and the organic phase was extracted with EtOAc (3×30 mL), washed with brine and dried over Na₂SO₄. Filtered through a short cotton pad and concentrated under vacuum. The crude was purified by flash column chromatography over SiO₂ using Hexane/EtOAc (gradient from 70% to 100%) as eluent. The product was isolated as a 3:1 mixture of diastereomers. Yellow oil (130.0 mg, 82% yield, [95% purity based on NMR]. Major isomer: ¹H NMR (500 MHz, CDCl₃) δ3.87 (s, J=1.7 Hz, 1 H), 3.68-3.55 (m, 2 H), 2.50 (s, 2 H), 1.80-1.71 (m, 1 H), 1.66-1.50 (m, 4 H), 1.50-1.30 (m, 6 H), 1.29-1.18 (m, 2 H). ¹³C NMR (126 MHz, CDCl₃) δ69.1, 63.0, 41.2, 33.0, 30.0, 27.9, 27.2, 25.1, 20.8. Minor isomer: The ¹H NMR signals are all overlaped with the exception of ¹H NMR (400 MHz, CDCl₃) δ3.23 (td, J=9.5, 4.5 Hz, 1 H), 1.98-1.91 (m, 1 H). ¹³C NMR (101 MHz, CDCl₃) δ74.8, 63.3, 44.8, 36.0, 30.6, 29.7, 28.4, 25.7, 25.1.

1. A process for producing an organic compound A, the process comprising:
reacting a compound B with hydrogen and water in the presence of at least one homogeneous transition metal catalyst TMC 1 in a single process step,
wherein the organic compound A is a compound of the formula (I) 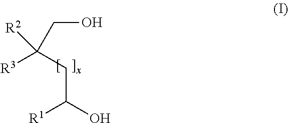
wherein
R¹ is an organic radical having from 1 to 40 carbon atoms,
R² is hydrogen or an organic radical having from 1 to 40 carbon atoms, and
R³ is hydrogen or an organic radical hvying from 1 to 40 carbon atoms, or
wherein R¹ together with R³ or R² together with R³, together with the atoms connecting them, form a divalent organic group having from 1 to 40 carbon atoms, and
wherein x is an integer from 1 to 10;
wherein the compound B is a compound of the formula (II) 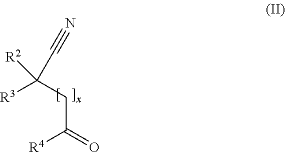
wherein
R² is hydrogen or an organic radical having from 1 to 40 carbon atoms,
R³ is hydrogen or an organic radical having from 1 to 40 carbon atoms, and
R⁴ is an organic radical having from 1 to 40 carbon atoms, or
wherein R⁴ together with R³ or R² together with R³, together with the atoms connecting them, form a divalent organic group having from 1 to 40 carbon atoms, and
wherein x is an integer from 1 to 10.

2. The process according to claim 1, wherein the homogeneous transition metal catalyst TMC 1 comprises a transition metal selected from the group consisting of metals of groups 8, 9 and 10 of the periodic table of the elements according to IUPAC.

3. The process according to claim 1, wherein the homogeneous transition metal catalyst TMC 1 is selected from the group consisting of [Ru(PPh₃)₃(CO)(H)Cl], [Ru(PPh₃)₃(CO)Cl₂], [Ru(PPh₃)₃(CO)(H)₂], [Ru(binap)(Cl)₂], [Ru(PMe₃)₄(H)₂], [Ru(PEt₃)₄(H)₂], [Ru(Pn-Pr₃)₄(H)₂], [Ru(Pn-Bu₃)₄(H)₂], [Ru(Pn-Octyl₃)₄(H)₂], [Ru(Pn-Bu₃)₄(H)₂], [Ru(PnOctyl₃)₄(H)₂], [Ru(PPh₃)₃(CO)(H)Cl], and [Ru(PPh₃)₃(CO)(H)₂].

4. The process according to claim 1, wherein the homogenous transition metal catalyst TMC_1 is used in an amount of 0.001 mol % to 20 mol %, calculated as transition metal and based on the amount of compound B used in the process.

5. The process according to claim 1, wherein the reaction between compound B, water and hydrogen is performed at a pressure in the range from 20 to 180 bar.

6. The process according to claim 1, wherein the reaction between compound B, water and hydrogen is performed at a temperature in the range from 50° C. to 180° C.

7. The process according to claim 1, wherein the reaction between compound B, water and hydrogen is performed in the presence of at least one solvent selected from the group consisting of dioxane, tetrahydrofuran, glymes, methanol, and ethanol.

8. The process according to claim 1, wherein the homoueneous transition metal catalyst TMC 1 is recycled by removing the compound A and other volatile compounds of the reaction mixture via distillation.
